(19) United States
(12) Patent Application Publication
(10) Pub. No.: US 2018/0093047 A1
(43) Pub. Date: Apr. 5, 2018

(54) SAFE NEEDLE DEVICE FOR SYRINGES

(71) Applicant: Dietrich Walter Grabis, San Rafael, CA (US)

(72) Inventor: Dietrich Walter Grabis, San Rafael, CA (US)

(73) Assignee: ITG-MEDICAL, INC., San Rafael, CA (US)

(21) Appl. No.: 15/285,671

(22) Filed: Oct. 5, 2016

(51) Int. Cl. A61M 5/32; A61M 5/315

(52) U.S. Cl. CPC A61M 5/3234; A61M 2005/31516; A61M 5/31513

(57) ABSTRACT

A self-retracting safety syringe for single use, wherein a needle receiver and retractor, located inside the hollow cavity of the syringe plunger, eject the needle into the plunger, which slides frictionless and freely inside the syringe barrel to facilitate ease of one-hand operation during aspiration of liquids and during injection whereby the tip of the plunger has mating surfaces to the needle base so that dead space is eliminated, which assures the clinician of administering the correct dosage of medication to the patient.

### CROSS REFERENCE TO RELATED APPLICATION

The present application is a continuation in part of U.S. Ser. No. 14/853,158 filed on Sep. 9, 2015 and of U.S. Ser. No. 13/723,258 filed on Dec. 21, 2012 entitled “Safe Needle Device for Syringes” the entire disclosure of which is incorporated by reference herein.

### BACKGROUND OF THE INVENTION

### 1. Field of Invention

This invention relates to safe hypodermic needles wherein the needle is protected after injection to prevent medical practitioners from receiving needle stick injuries and infections.

### 2. Description of Related Art

The present invention is directed in general to a safe needle device for syringes as used in the medical industry and hospitals. The “Improvement patent” (classified as UTILITY patent, according to the USPTO) application presents significant improvements, which are based upon numerous marketability tests (see Commercial Feasibility after the Background section below) conducted over the past several years and after FDA 510(k) approval to market the invention, issued under K010477. The improvements are:
a) Reducing the bulkiness of the device as expressed by Vanderbilt University's Medical School of Nursing, Nashville, Tenn. and other potential users.
b) Providing an ergonomically more convenient location and use of the retraction release.
c) Reducing dead space to eliminate wasting medication and assuring proper dosage to the patient.
d) Reducing plunger friction during aspiration to insure easy one-hand operation by clinicians, doctors and nurses.
e) Simplifying spring retention and thereby assuring reliable retraction of the needle.
f) Eliminating components inside the syringe barrel and plunger to permit low-cost manufacturing.
g) configuring a needle retraction path so that no blood is left outside the syringe after retraction to avoid contamination or infection of the administering clinician.
h) Assuring the appearance of a conventional syringe that clinicians are familiar with by showing a rubber seal at the tip of the plunger through a clear syringe body.

With the advent of AIDS and other highly infectious diseases, syringes and scalpels and other sharps devices have caused numerous injuries and infections to administering medical staff. Protective devices have been introduced to the market after the U.S. legislation in 1998, but consisted of needle covers that still left part of the needle exposed after use and thereby subjecting the clinician to exposure to contaminated blood.

These devices are still on the market, but are not user friendly and safe. For example, a nurse must use two hands to prepare a patient for an injection. One hand is used to clean the area to be injected with a disinfectant while the other hand is used to inject the needle into the tissue. After removing the needle from the patient, the administering nurse must press on the spot of injection and hold the syringe in the other hand. After pressing the spot is complete (to stop the flow of blood), the nurse, in prior protective devices, then uses that hand to twist a cover over the syringe or sharp. That requires two hands during which time the needle may drip blood on the patient or the nurse where contamination is inevitable. In addition, the maneuvering to accomplish the foregoing may cause an inadvertent puncturing of the nurse's own skin, thus inviting infection. One such device is Badger U.S. Pat. No. 5,885,257, which is a very cumbersome releasable retaining device to retract the needle where it will not cause harm.

In 2012 the WHO (World Health Organization) in Geneva, Switzerland, published a decree saying that all countries must implement the use of RETRACTABLE safety syringes by the year 2020. That means conventional needle covers, which don't provide the needed protection, will no longer be acceptable or allowed.

In this Improvement patent filing the above statements remain unchanged, but the ease of handling during administering medication by a clinician is greatly improved and so is increased safety to the patient and clinician. Two mechanically retracting safety syringes came on the market, i.e. the Integra, marketed by BD (Becton Dickenson) of Franklin, N.J., U.S. Pat. No. 5,632,733 (Shaw) and U.S. Pat. No. 7,351,224 (Shaw) and the Vanish Point, marketed by RTI (Retractable Technology, Inc.) of Houston, Tex., U.S. Pat. No. 6,090,077 (Shaw, 7/2000). While the spring-operated needle retraction is the same in both devices (BD and RTI) they both have their compression springs located in the front end of the syringe whereas the present invention has the spring located inside the plunger. In addition, the Vanish Point does not have a Luer-Lock and, thus, does not permit interchangeability of needles prior to use. The Integra is hard to push to cause retraction which is a detriment for single hand operation because it can cause significant patient discomfort especially when the device is triggered while the needle is still in the patient, The spring assembly in the Integra (BD) can easily be removed after retraction; it falls apart. The 3 patents cited above, I.e. U.S. Pat. No. 5,632,733, U.S. Pat. No. 7,351,224 and U.S. Pat. No. 6,090,077, issued to Shaw 9 years after Botich FIGS. 1-5, are identical in their basic construction and function to Botich, FIGS. 1-5. They all use what is referred to in the industry as “front retraction”, meaning that the spring is at the distal end of the syringe and is part of the needle base.

In contrast to Botich, FIGS. 1-5 and the Shaw inventions, Botich shows in FIG. 6 a rear retraction syringe where the needle retraction is activated from the proximal end of the syringe (see U.S. Pat. No. 4,994,034 FIG. 6, Botich (1991). There are many differences between Botich and the current invention. For example, Botich teaches the retraction of an injection needle via a spring inside a spring housing that is pushed into the hollow cavity of a syringe plunger that does not have a conventional rubber seal. Although similar, Botich achieves this by different means. Specifically, (claim 15 (b), lines 27-42), Botich states that the use of a “cylindrical spring housing . . . wherein a spring is retained, . . . having exteriorly located attachment tabs . . . ” retraction is accomplished.

The syringe housing is “ . . . held by said syringe barrel . . . ” (claim 16 (d), col. 14, lines 7-8). In contrast, the instant invention does not use a spring housing. Instead, the spring is retained against a collar inside the plunger without the need for holding means against the barrel, which, when combined with Botich's claim 15 (d), col. 12, lines 58-62, stating “ . . . sealing means to provide an air tight seal between said exterior of said cylindrical spring housing and said interior of said barrel . . . ”, creates excessive friction during aspiration of medication making one-hand operation very difficult, if not impossible.

Botich, furthermore, teaches that his spring housing, i.e. the needle retractor housing, upon “Further downward pressure on the plunger 59 forces the needle retractor housing 115 past detents 117 . . . ” (col. 9, lines 48-50), which (detents) by the way, are located outside the plunger on the inner wall of the syringe barrel, as mentioned before. This means that Botich's needle retractor housing, after it passes the external (to the plunger) detents 117 “ . . . is moved deeper into the cylindrical cavity 71 of the plunger 59.” (col. 9, lines 54-56). Botich does not explain how his needle retractor housing with external hooks on the plunger, that engage with detents 117, can “move deeper into the cylindrical cavity 71 of the plunger 59”. That restriction requires “ . . . an extra piston spacer 123” (col. 9, line 57). None of these constraints and requirements “ . . . for proper operation . . . ” (col. 9, line 57) exist in the instant invention. In claim 2 Botich states “ . . . said frangible end of said syringe plunger separates (to allow retraction) when a “ . . . force is exerted . . . ” (col. 10, lines 56-57). In other words without the break-off/separation, there is no retraction. No such separation takes place in the present invention, which assures retraction everytime because of an entirely different and superior receiver assembly design.

In claim 5 Botich states “ . . . wherein said interior cavity of said syringe plunger is evacuated prior to the separation of said frangible end . . . ” (col. 11, lines 5-6). The present invention does not require evacuation—which Botich needs to suck up spilled blood—; it is free of any blood to be spilled, which is a prerequisite of any needle safety device as mandated by the FDA/CDC. Botich could not meet today's requirements with his inferior design. The present invention meets all requirements of the FDA.

Furthermore, the distal end of Botich's needle retention housing is not conducive to reducing dead pace because of a mismatch in surfaces. The present invention addresses this aspect specifically by means of mating surfaces. Finally, Botich describes in claim 15 (d), col. 12, line 53 that the syringe barrel is “ . . . engaging and holding said cylindrical spring housing . . . ” by means of “ . . . slots and a groove . . . ” (col. 12, line 55) inside the barrel. The present invention does not require or use a spring housing and, consequently, does not require slots and a groove in the barrel.

In U.S. Pat. No. 5,053,010, McGary states in claim 1. “ . . . a cutting tip configured inside said sealing member . . . ” in claim 2 he states “ . . . said cutting tip is a beveled knife”.

The current invention does not use any cutting tip. It uses a mechanically interlocking tip-lock. In U.S. Pat. No. 5,211,629, Pressly (10/1991) states “Positioned between sacrificial supports in the needle assembly and the barrel is a deformable base and . . . the plunger has a rupturable boot”. The McGary and Pressly inventions are quite a departure from the Grabis invention.

After extensive marketability testing and commercial feasibility evaluation, the present invention was made faster in retraction, smaller in the outer diameter, lighter and cheaper to manufacture.

### SUMMARY OF THE INVENTION

A self-retracting safety syringe has a hollow syringe body, a hollow needle having a point at its tip and a tip-lock at its base, a sleeve around the needle configured to sealingly retain the needle, a hollow plunger configured to engage the interior wall of the syringe body, the plunger having a tip sealingly engaged with the interior wall and having an aperture therein to accommodate the tip-lock, a collar circumferentially mounted on the interior wall of the plunger, a retractor for accepting and retaining the tip-lock, wherein the distal end is retained within the proximal end of the plunger by the collar, and a spring biasing the retractor against the collar, wherein the tip-lock and plunger join, and wherein the retractor is pushed past the collar and is adapted to pull the needle from within the sleeve into the hollow plunger.

In an embodiment, the sleeve has a Luer-Lock connection at the distal end of the syringe body to sealingly retain the needle against the syringe body.

The foregoing and other features and advantages of the invention will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a cross-sectional view of a syringe embodying the improved invention showing its basic standard syringe construction with a female Luer-Lock connection and vapor lock.

FIG. 2 is a cross-sectional view of the needle base assembly with needle connection means to allow the needle to disappear inside the plunger after activation.

FIG. 3 shows a cross-sectional view of the spring retention means inside the plunger and the compressed spring in the retention means at the forward end of the plunger.

FIG. 4 shows a side view of the syringe and the plunger with an end plug.

FIG. 5 shows an exploded view of the syringe according to an embodiment of the present Invention.

### DETAILED DESCRIPTION OF PREFERRED EMBODIMENTS

Preferred embodiments of the present invention and their advantages may be understood by referring to FIGS. 1-5).

It is a general object of the improvements to the original invention, as shown in patent U.S. Pat. No. 6,322,540 B1 to provide a further improved retractable safety needle device for syringes.

In accordance with the above object, there is provided a needle-stick safety syringe comprising a tubular body, cylindrical plunger, and an extended needle at the distal end of the tubular body. Said needle to be removable, through attachment means, such as a Luer-Lock connector, in order to be replaced with a different size needle if so desired or required based upon patient needs or with a blunt fill needle or tubing with matching connectors.

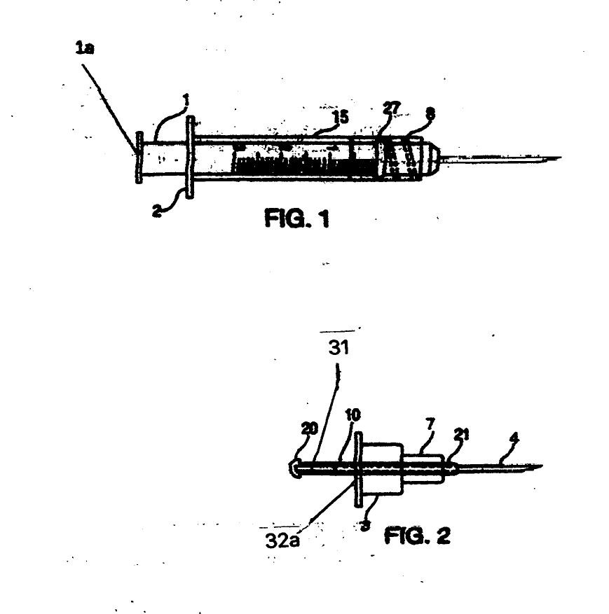

Referring now to FIG. 1, a typical commercial syringe that has a small (for example, 3 ml) capacity and is illustrated with a tubular body 15 and plunger 1 having a connected rubber seal 27 that slides within the tubular body 15 and sealingly and slidingly engages the inside wall of the tubular body 15. The plunger 1 has a flat end 1a adapted to be pushed by the thumb. The tubular syringe body 15 has a flange 2 extending outwardly at its proximal end for engagement with the fingers to permit one-handed operation. The distal end 8 of the tubular body 15 has an extended female Luer-Lock screw-on connector 8 which is an integral part of the syringe body 15. The connector 8 comprises a cylinder projecting from the distal end of the tubular body 15 having internal threading for accepting a needle thereon. Within the cylinder is a tapered nozzle which sealingly engages with the needle when the needle is engaged. With reference to FIG. 2 the needle base assembly 3 is adapted to be inserted into the connector 8 and is retained there.

The needle and sleeve are mounted within the female connector 8.

FIG. 2 shows a needle base assembly 3 providing means to permit extracting the needle 4 from a sleeve 7. The needle 4 is encased in a bonding plastic material within the sleeve 7 at its distal end, as is done with all syringes whether they are safety syringes or standard syringes, and then connected to a retainer 10 providing connecting means to the encased needle 4 through a tip-lock 20 that will engage in the receiver in the plunger 1. The retainer 10 is wedged into the sleeve 7 via a conically shaped collar 21; alternatively, a sealant may be used.

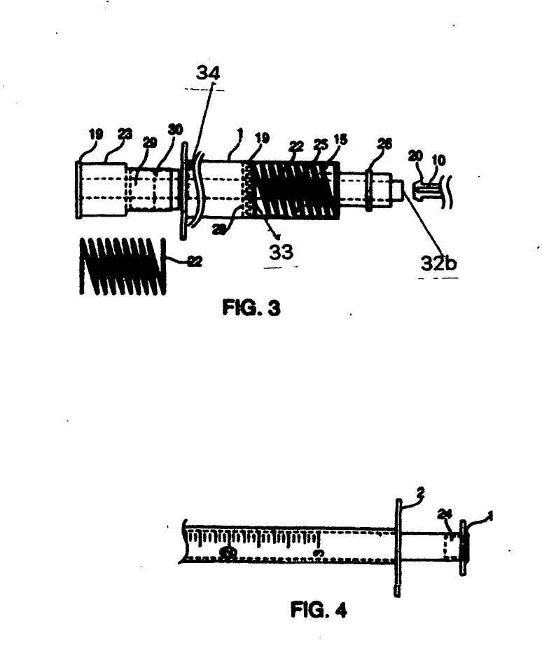

FIG. 3 shows the plunger 1 that houses the compression spring 22. A receiver assembly 23 and 29, permitting the retraction of the needle 4, provides the interconnection between the distal end 26 of the plunger 1 and the needle base assembly 3 having a tip-lock 20 having a cylindrical body inside a cylindrical adapter FIG. 2, 7. Said needle base assembly 3 having a male thread at its distal end in form of a ring (FIG. 2) that comprises a single male thread that provides means to engage it into a mating female thread. Said female thread is at the distal end of the syringe body and comprises a Luer-Lock connector. Said cylindrical body inside said cylindrical adapter performs a dual function. It provides the means to firmly encase the base of the needle 4 and is shaped on the opposite end to provide means to engage the receiver assembly 23 and 29 inside the plunger 1.

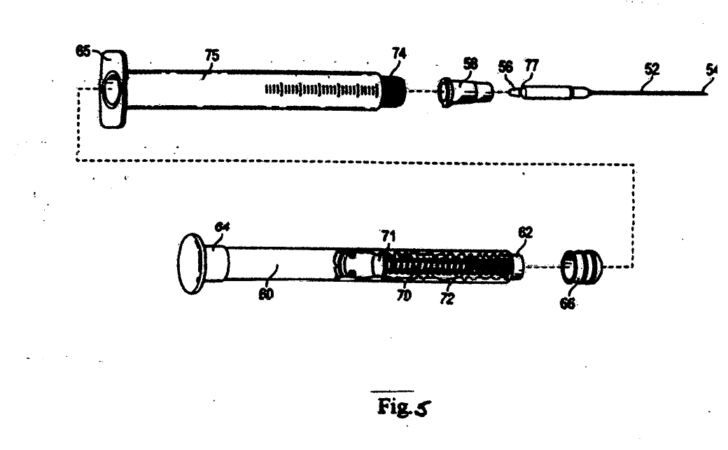

The compression spring 22 is positioned inside said plunger 1 between a collar 19 and an Inward lip (FIG. 3, 15) at the distal end of the plunger 1 providing spring retention means and the means to interconnect said plunger 1 with said tip-lock 20. Both retention points are located inside the plunger 1. The needle receiver assembly 23 and spring 22 are sized to pass the needle 4 with its needle base assembly (FIG. 2, 7). A stop (FIG. 4, 24 and FIG. 5, 64) inside said plunger 1 is limiting further expansion of said spring 22 after said needle extraction is activated. Through the force of the uncoiling compression spring 22 in the plunger 1, the needle 4 is pulled out of the sleeve 7 (FIG. 2) and retracted fully through the hole in the rubber seal (FIG. 5, 66) and the inside of the spring 22 into the plunger 1. The needle receiver assembly 23 and 29 and spring 22 are sized to pass the needle 4 and its sleeve 7 through the rubber seal 66 and through the spring during retraction.

The tip-lock 20 engages with the plunger 1 and locks to the plunger tip, through the rubber seal 66 and the spring 22 to the receiver assembly 23 and 29 inside the plunger 1. The plunger tip and needle base assembly (FIG. 2) have mating surfaces so that dead space can be eliminated and the loss of medication be minimized to assure proper dosage to the patient. Slots in the tip-lock 20 raid in the dispensing of liquids.

The spring 22 serves to retract the needle 4 when activated by the tip-lock 20, which pushes the receiver assembly 23 and 29 through a friction collar 28 after the tip-lock 20 is connected to the tip of the plunger 1. The spring 22 is compressed between the tip of plunger 1 and a collar 19 within the plunger 1 so that the spring 22 can not slide over it while compressed. As the spring 22 uncoils it pushes said receiver assembly 23 and 29 and the locked tip-lock 20, with needle 4 attached to it, to the proximal end of the plunger 1. The wedged pressure-fitted collar 28, being positioned on the inside wall of the plunger 1, permits the tip-lock 20 to push the collar 19, which is part of the receiver assembly 23 and 29, through it, by applying thumb pressure to the end of the plunger 1 so that the spring 22 can uncoil and pull the receiver assembly 23 and 29 with its connected needle 4 with sleeve 7 out of the needle base assembly (FIG. 2) and into the inside of the plunger 1; the sleeve 7 of the needle base assembly (FIG. 2) with needle 4 are being retracted through the inside of the rubber seal 66 and the inside of the spring 22.

As FIG. 3 illustrates, the plunger 1 has no obstructive protrusions on its outside wall, thereby permitting frictionless and free movement of the plunger 1 inside the syringe barrel with resulting ease of one-hand operation by a clinician during aspiration of liquids and during injection providing maximum comfort to the patient.

FIG. 4 shows a plug 24 inserted into the proximal end of plunger 1 by means of a pressure fitting or sonic welding. It prevents the retracted needle 4 from coming out of the back of the plunger 1 after retraction, however, the spring also slows expansion naturally as it uncoils.

With reference to FIG. 5, in another embodiment the needle extraction mechanism is shown in an exploded view. The needle 52 is hollow for delivering fluids and has a point 54 at the proximal end and a tip-lock 56 at the distal end. The tip-lock is affixed around the needle 52 and extends therefrom and is sealingly and frictionally engaged within the male Luer-Lock sleeve 58. The sleeve 58 is adapted to engage with the complimentary Luer-Lock 74 of the syringe cylinder 75. The syringe cylinder is open at the distal end to accept the plunger 60, and it is flared 65 for engagement with the fingers to depress the plunger. Upon the application of force, the tip-lock 56 and attached needle 52 may be removed from within the sleeve 58. The needle 52 is seated within the sleeve 58, and in an embodiment, sealed therein. The needle 52 is retained within the sleeve such that it may not proceed through the sleeve 58 in the distal direction (the direction of the patient), however, it is easily retractable into the proximal direction (away from the patient). In an embodiment, the needle 52 is sealed with a sealant.

With further reference to FIG. 5, the plunger 60 is cylindrical and hollow and contains a collar therein. The plunger 60 is adapted to fit within the syringe cylinder 75. The plunger 60 has a distal end 62 having a rubber seal 66 that sealingly engages the wall of the cylinder, and has a proximal end 64 that is flattened perpendicularly, adapted for engagement with a thumb to push the plunger 60 into the cylinder. The rubber seal 66 has an aperture (center hole not shown) therein providing access to a retractor 70. The retractor 70 is positioned beneath the collar, wherein the retractor 70 is kept within the distal end 62 of the plunger 60. A spring 72 is contained between the distal end 62 and the retractor 70, and biased the retractor 70 against the collar and towards the proximal end 64. The retractor 70 has a collar 71 to engage with the spring 72 from passing over the collar 71. On application of force, the retractor 70 bypasses the collar and is released into the proximal, closed end 64 of the plunger 60, pushed by the spring 72. The end plug 64 retains the retractor 70 within the proximal end of the plunger 60.

In use, as the plunger 60 descends, the rubber seal 66, attached to the distal end 62 of the plunger 60, sealingly pressurizes liquids within the cylinder 75 such that they are expelled out the needle 52, typically into a patient. The needle 52 has a channel therein to permit the passage of fluids therethrough. The tip-lock 56 is mounted on supports 77 to permit fluid to flow through the rubber seal 66, which has an aperture therein (center hole not shown) to permit access to the retractor 70. As the plunger 60 is depressed, the tip-lock 56 enters the retractor 70 and is locked therein. At the distal end, the retractor 70 has a receiver adapted to receive and retain the tip-lock 56, and a stop to prevent the tip-lock 56 from moving through the retractor 70. As the plunger 60 is further depressed, the tip-lock 56 passes the retractor 70 opening through the rubber seal 66 and the center of the spring 72 and engages with the receiver inside the plunger 60. The force causes the distal end of the retractor 70 to push against the collar, and move past the collar to enter the proximal end of the interior of the plunger 60. As the retractor 70 is spring biased into the distal end of the plunger 60, it moves with some rapidity into the end, pulling the tip-lock 56, and consequently the needle 52, along with it into the body of the plunger 60.

The invention has been described herein using specific embodiments for the purposes of illustration only. It will be readily apparent to one of ordinary skill in the art, however, that the principles of the invention can be embodied in other ways. Therefore, the invention should not be regarded limited in scope to the specific embodiments disclosed herein, but instead as being fully commensurate in scope with the following claims:

1. A self-retracting safety syringe comprising:
(a) A syringe barrel consisting of a tubular body;
(b) A single component tubular plunger including an end plug and sized to fit into the syringe barrel in a sliding liquid sealing configuration;
(c) The said plunger being hollow and comprising a single component tubular body without the use of a plunger housing or disengageable plunger member;
(d) An injection needle;
(e) A needle base assembly located at the proximal end of the needle and providing connecting means to a receiver assembly;
(f) Said receiver assembly located inside the distal end of said single component tubular plunger provides connecting means to the said needle base assembly;
(g) A spring retention means, including a retention slip ring, for holding a spring inside said plunger in a compressed configuration for retraction of the needle with its said needle base assembly into said interior cavity of said single component tubular plunger through thumb pressure applied to said connecting receiver assembly, which allows the said receiver assembly to slip through the retention slip ring and thereby causing the spring, located inside said single component tubular body of the plunger and against the inner wall of said plunger, to uncoil;
(h) A rubber seal with a mating surface to the said needle base assembly to minimize dead space;
(i) A said needle base assembly having vertical openings in its connector to permit liquid to pass through it;

2. A safety syringe system as claimed in claim 1, wherein said spring retention means cause the spring to uncoil and pass through a retention slip ring inside the said single component tubular body of said plunger after applying thumb pressure to said plunger and thereby retracting said needle;

3. A safety syringe system as claimed in claim 2, wherein said spring retention means are located inside said single component tubular body of the plunger and located in the center thereof;

4. A safety syringe system as claimed in claim 3, wherein said spring retention means are sized to permit passage through said retention slip means inside said single component tubular body of the said plunger;

5. A safety syringe system as claimed in claim 4 wherein said receiver assembly is sized to pass the said needle with its said needle base assembly through the said spring when the retraction is activated.

6. A safety syringe system as claimed in claim 5, wherein the said receiver assembly, in conjunction with the distal end of the said rubber seal, is configured to prevent dead space after completing the injection and thereby minimizing wasting medication and assuring the correct dosage to the patient;

7. A safety syringe system as claimed in claim 6, wherein said rubber seal has a hole in its center and sealingly engages the wall of the said syringe barrel;

8. A safety syringe system as claimed in claim 7, wherein said hole in said rubber seal is sufficiently in diameter to allow said needle with its needle base assembly to pass through it when the retraction of said needle is activated;

9. A safety syringe system as claimed in claim 8, wherein the said single component tubular plunger, being located in the center of the barrel, has a compressible spring inside its tubular cavity that is positioned against the inside wall of said single component tubular plunger;

10. A safety syringe system as claimed in claim 9, wherein the said needle base has vertical slots in its connecting means to permit fluid to pass through it to aid in the elimination of dead space in conjunction with the mating surfaces of the said receiver assembly inside the said single component tubular plunger.
